(19) United States
(12) Patent Application Publication
(10) Pub. No.: US 2014/0284222 A1
(43) Pub. Date: Sep. 25, 2014

(54) Detection Of Synergistic And Additive Trace Gases

(71) Applicants: Richard William Wanek, JR., Pittsburgh, PA (US); Greg Martin Sesny, Pittsburgh, PA (US)

(72) Inventors: Richard William Wanek, JR., Pittsburgh, PA (US); Greg Martin Sesny, Pittsburgh, PA (US)

(73) Assignee: Draeger Safety, Inc., Pittsburgh, PA (US)

(21) Appl. No.: 14/355,809

(22) PCT Filed: Nov. 1, 2011
(86) PCT No.: PCT/US2011/058749

§ 371 Date: May 1, 2014

(51) Int. Cl. G01N 33/00; G01N 21/61; G01N 27/64; G01N 27/26

(52) U.S. Cl. CPC G01N 33/0065; G01N 27/26; G01N 21/61; G01N 27/64
USPC 205/780.5; 204/406; 205/775; 204/431; 250/343; 250/340; 250/336.1; 250/395; 422/83; 702/24

(57) ABSTRACT

Described herein is a gas meter system (5) for detecting toxic gas combinations including a housing (10) having an opening (20), a measuring cell (15) enclosed by the housing and comprising a plurality of gas sensors (17) in fluid communication with the environment via the opening in the housing, and an evaluating circuit (32). The gas sensors detects a concentration of a first gas and a concentration of a second gas so the evaluating circuit can identify a hazard due to an additive or synergistic toxic effect upon combined exposure to the first gas and the second gas. Related apparatus, systems, methods and/or articles are described.

The subject matter described herein relates to gas detection, monitoring and environmental warning systems.

### BACKGROUND

Electronic gas meters monitor hazardous concentrations of substances in an environment. Gas meters typically include one or more gas sensors and electronics to convert an output signal from the sensors into one or more signals representative of the gas concentration being monitored. The gas meters can detect levels of the individual toxins beyond a defined level considered to be harmful and then generate a warning.

Combined exposure of some gases or toxins can have an additive or synergistic toxic effect that at an individual level would not have been considered harmful or requiring a call to action.

### SUMMARY

In one aspect, disclosed is a gas meter system for detecting toxic gas combinations. This system includes a housing having an opening; a measuring cell enclosed by the housing and having a plurality of gas sensors in fluid communication with the environment via the opening in the housing, and an evaluating circuit. The gas sensors detect a concentration of a first gas and a concentration of a second gas so the evaluating circuit can identify a hazard due to an additive toxic effect upon combined exposure to the first gas and the second gas.

The additive toxic effect can be identified by a Hazard Quotient (HQ) equation: HQ=C_{G1}/TLV_{G1}+C_{G2}/TLV_{G2} wherein C_{G1} is the first gas concentration; TLV_{G1} is a desired threshold limit value of the first gas; C_{G2} is the second gas concentration; and TLV_{G2} is a desired threshold limit value of the second gas. A hazard can be identified when the HQ is greater than or equal to a defined hazard limit. The hazard can be identified when at least one of the first gas concentration and the second gas concentration is below its respective defined threshold limit value. The hazard can be identified when both the first gas concentration and the second gas concentration are below their respective defined threshold limit values. The first gas and the second gas can be different gases. The first gas can be carbon monoxide and the second gas can be hydrogen cyanide. One or more of the gas sensors can be selected from the group consisting of an electrochemical, infrared, semiconductor, catalytic, photoionization, and galvanic sensor. The system can further include an alarm system to be activated when the hazard is identified. The alarm system can include at least one of an audible, visual, or tactile alarm.

In an interrelated aspect, a gas meter system for detecting toxic gas combinations is described including a housing having an opening, a measuring cell enclosed by the housing, a plurality of gas sensors in fluid communication with the environment via the opening in the housing, and an evaluating circuit. The gas sensors detect a concentration of a first gas and a concentration of a second gas. The evaluating circuit can identify a hazard due to a synergistic toxic effect upon combined exposure to the first gas and the second gas identified by a Hazard Quotient (HQ) equation: HQ=A*(C_{G1}/TLV_{G1})+B*(C_{G2}/TLV_{G2}), wherein A and B are multipliers; C_{G1} is the first gas concentration; TLV_{G1} is a desired threshold limit value of the first gas; C_{G2} is the second gas concentration; and TLV_{G2} is a desired threshold limit value of the second gas.

In an interrelated aspect, disclosed is a method of detecting an additive effect of toxic gases including sensing a concentration of a first gas in an environment using a gas meter system, sensing a concentration of a second gas in the environment using the gas meter system, providing the sensed concentrations of the first and second gases to the microprocessor, and using a microprocessor to calculate a Hazard Quotient (HQ) using equation: HQ=C_{G1}/TLV_{G1}+C_{G2}/TLV_{G2} wherein C_{G1} is the concentration of the first gas, TLV_{G1} is a first desired threshold limit value of the first gas, C_{G2} is the concentration of the second gas, and TLV_{G2} is a second desired threshold limit value of the second gas. Thereafter, it is evaluated whether the HQ is greater than or equal to a defined hazard limit programmed into the microprocessor.

The method can further include identifying a hazard when the HQ is greater than or equal to the defined hazard limit. The hazard can be identified when the concentration of at least one of the first gas and the second gas is below its respective defined threshold limit value. The hazard can be identified when both the first gas concentration and the second gas concentration are below their respective defined threshold limit values. The first gas and the second gas can be different gases. The first gas can be carbon monoxide and the second gas can be hydrogen cyanide. Sensing a concentration of the first gas can include using one or more gas sensors selected from the group consisting of an electrochemical, infrared, semiconductor, catalytic, photoionization, and galvanic sensor. Sensing a concentration of the second gas can include using one or more gas sensors selected from the group consisting of an electrochemical, infrared, semiconductor, catalytic, photoionization, and galvanic sensor. The method can further include activating an alarm system when the hazard is identified. The alarm system can include at least one of an audible, visual or tactile alarm.

In an interrelated aspect, disclosed is a gas measuring system including a first gas meter device configured to detect a concentration of a first gas within a monitored zone, a second gas meter device separate and apart from the first meter device configured to detect a concentration of a second gas in the monitored zone, and a base station in communication with the first gas meter device and the second gas meter device. The base station can calculate an additive toxic effect by a Hazard Quotient (HQ) equation: HQ=C_{G1}/TLV_{G1}+C_{G2}/TLV_{G2} wherein C_{G1} is the first gas concentration; TLV_{G1} is a desired threshold limit value of the first gas; C_{G2} is the second gas concentration; and TLV_{G2} is a desired threshold limit value of the second gas. The base station can also initiate an alarm when the additive toxic effect is above a pre-defined hazard limit.

In an interrelated aspect, disclosed is a method including sensing a concentration of a first gas in an environment using at least one gas meter system, sensing a concentration of a second gas in the environment using at least one gas meter system, calculating, based on the sensed concentrations of the first and second gases, a Hazard Quotient (HQ) using equation: HQ=C_{G1}/TLV_{G1}+C_{G2}/TLV_{G2} wherein C_{G1} is the concentration of the first gas; TLV_{G1} is a first desired threshold limit value of the first gas; C_{G2} is the concentration of the second gas; and TLV_{G2} is a second desired threshold limit value of the second gas, and initiating an alarm when the HQ is greater than or equal to a pre-defined hazard limit.

In an interrelated aspect, disclosed is a method of detecting a synergistic effect of toxic gases, including sensing a concentration of a first gas in an environment using a gas meter system wherein the gas meter system includes a microprocessor having a programmed first desired threshold limit value for the first gas; sensing a concentration of a second gas in the environment using the gas meter system, wherein the microprocessor of the gas meter system has a programmed second desired threshold limit value of the second gas; providing the sensed concentrations of the first and second gases to the microprocessor; using the microprocessor to calculate a Hazard Quotient (HQ) using equation: HQ=A*(C_{G1}/TLV_{G1})+B*(C_{G2}/TLV_{G2}) wherein A and B are multipliers; C_{G1} is the first gas concentration; TLV_{G1} is a desired threshold limit value of the first gas; C_{G2} is the second gas concentration; and TLV_{G2} is a desired threshold limit value of the second gas; and evaluating whether the HQ is greater than or equal to a defined hazard limit programmed into the microprocessor.

In an interrelated aspect, disclosed is an article including computer executable instructions permanently stored on non-transitory computer readable media, which, when executed by at least one data processor, causes the at least one data processor to perform operations including: receiving first data characterizing sensing of a concentration of a first gas in an environment using a gas meter system, the first gas having an associated first desired threshold limit value; receiving second data characterizing sensing of a concentration of a second gas in the environment using the gas meter system, the second gas having an associated second desired threshold limit value; calculating a Hazard Quotient (HQ) using equation: HQ=C_{G1}/TLV_{G1}+C_{G2}/TLV_{G2} wherein C_{G1} is the concentration of the first gas; TLV_{G1} is the first desired threshold limit value of the first gas; C_{G2} is the concentration of the second gas; and TLV_{G2} is the second desired threshold limit value of the second gas; and identifying a hazard when the HQ is greater than or equal to a pre-defined hazard limit.

In an interrelated aspect, disclosed is an article including computer executable instructions permanently stored on non-transitory computer readable media, which, when executed by at least one data processor, causes the at least one data processor to perform operations including: receiving first data characterizing sensing of a concentration of a first gas in an environment using a gas meter system, the first gas having an associated first desired threshold limit value; receiving second data characterizing sensing of a concentration of a second gas in the environment using the gas meter system, the second gas having an associated second desired threshold limit value; calculating a Hazard Quotient (HQ) using equation: HQ=A*(C_{G1}/TLV_{G1})+B*(C_{G2}/TLV_{G2}) wherein A and B are multipliers; C_{G1} is the concentration of the first gas; TLV_{G1} is the first desired threshold limit value of the first gas; C_{G2} is the concentration of the second gas; and TLV_{G2} is the second desired threshold limit value of the second gas; and identifying a hazard when the HQ is greater than or equal to a pre-defined hazard limit.

Articles of manufacture are also described that comprise computer executable instructions permanently stored on non-transitory computer readable media, which, when executed by a computer, causes the computer to perform operations herein. Similarly, computer systems are also described that may include a processor and a memory coupled to the processor. The memory may temporarily or permanently store (e.g., non-transitorily store, etc.) one or more programs that cause the processor to perform one or more of the operations described herein. In addition, methods described herein can be implemented by one or more data processors either within a single computing system or distributed among two or more computing systems.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a block diagram of an implementation of a gas meter system.

FIG. 2 is a schematic of an implementation of a gas meter system.

FIG. 3 is a schematic of an implementation of a plurality of gas meter systems communicating with a base station.

FIG. 4 is a flow diagram of an implementation of a method of use of a gas meter system.

FIG. 5 is an exploded view of an implementation of a gas meter system.

Like reference symbols in the various drawings indicate like elements.

### DETAILED DESCRIPTION

Disclosed herein are devices, systems and methods to analyze concentrations of substances, such as gas species, that alone or individually would not normally require a call to action, but in combination with another may present a threat. The devices, systems and methods described herein incorporate algorithms that can identify potential synergistic or additive effects of more than one gas species and the potential danger of these gas species acting in combination. Data collected by the devices (sometimes referred to herein as metering systems) can be used to calculate a danger level based on the new defined threshold and indicate that level to those in the area to alert them to take appropriate action.

When toxic gases combine, they can have an additive effect or synergistic effect. An additive effect occurs when the combination of two substances produces a total effect that is equal to the sum of the individual effects. Thus, toxicity of these substances can be determined through addition. A synergistic effect occurs when the combination of two substances produces a total effect that is greater than the sum of the individual effects. Toxicity of these substances is exponential.

Carbon monoxide (CO) and Hydrogen Cyanide (HCN) are examples of toxic substances that can have additive toxicity. The mixture of CO and HCN can be measured by a Hazard Quotient (HQ) equation:

HQ=C_{co}/TLV_{co}+Cₕₑₙ/TLVₕₑₙ
where C_{co}=Concentration (CO); TLV_{co}=Desired threshold limit value (CO);
Cₕₑₙ=Concentration (HCN); and TLVₕₑₙ=Desired threshold limit value (HCN).

If HQ≧1, then a potential hazard exists. Individually, the limit for CO can be as low as 25 ppm and the limit for HCN can be 4.7 ppm. Because of the additive toxicity for CO and HCN if the substances are present in combination a potential hazard can exist although one or both of the substances are below their normal individual limits. For example, if the concentration of CO is 20 ppm (just below the limit for CO) and the concentration of HCN is 1 ppm (well below the limit for HCN), the HQ calculation is: HQ=20 ppm/25 ppm+1 ppm/4.7 ppm=0.8+0.21=1.01. Because the resulting HQ is ≧1, the combination of these substances at these concentrations can result in an alarm even though neither substance individually is above its threshold limit. The devices and systems described herein generate a more specific and reliable alarm for gas detection by detecting combination of specific events. The threshold algorithms incorporated in the gas meters described herein can accurately measure the ambient gas mixtures and use accepted exposure limits of the individual gases to calculate the danger potential and establish the extent to which toxicity is increased and whether or not to provide a warning.

This same rationale can be applied to other combinations of substances. It should be appreciated that the gas combinations are not limited to the specific combinations identified herein and that almost any individual toxic gas acting with another toxic gas is considered herein. For example, a combination of more than two substances is considered herein. The specific calculation and equations are dependent on whether the effect is additive or synergistic. For example, a synergistic effect can include some type of multiplier in front of the individual TLVs depending on the combination of gases for example as shown in the Hazard Quotient (HQ) equation:

HQ=A*(Cₓ/TLVₓ)+B*(C_{y}/TLV_{y})
where
A and B are multipliers; Cₓ=Concentration (gas x); TLVₓ=Desired threshold limit value (gas x); C_{y}=Concentration (gas y); and TLV_{y}=Desired threshold limit value (gas y).

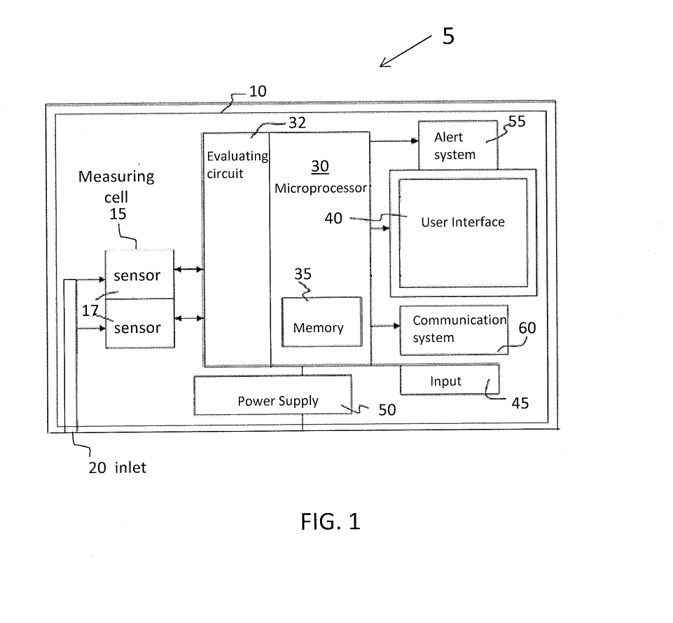

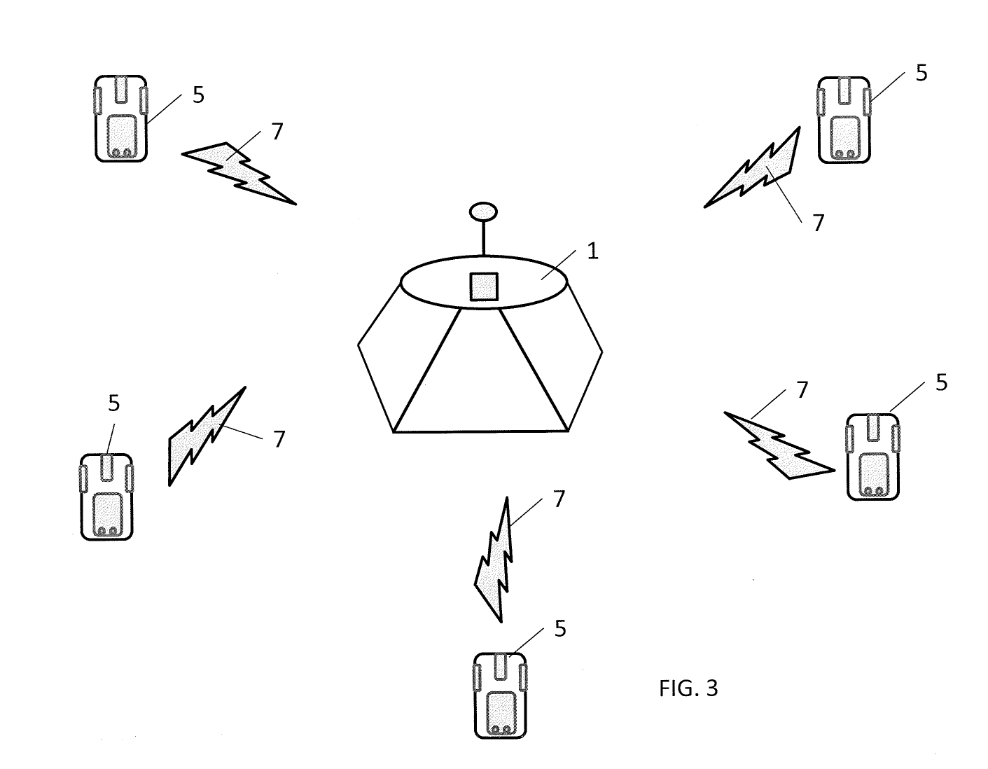

FIG. 1 is a schematic of an implementation of a gas meter system 5. The gas meter system 5 can be used in disciplines where a combination of individual events can be detected to prompt an action or reaction. One or more gas meter systems 5 can be used in combination with an area monitoring device 1 (see FIG. 3) or independently in areas where gas hazards are likely to occur. In some implementations, the meter system 5 can be used in patient monitoring, patient transport and mobility, ventilation therapy, wireless care, firefighting, confined space entry such as shafts, tunnels or tanks, alcohol ignition interlock, and others. The systems described herein can have application in any area where toxic gases may occur, such as military and law enforcement use as well as in hospitals, research facilities, and industrial facilities to detect exposure to dangerous substances that might be inadvertently released into the environment.

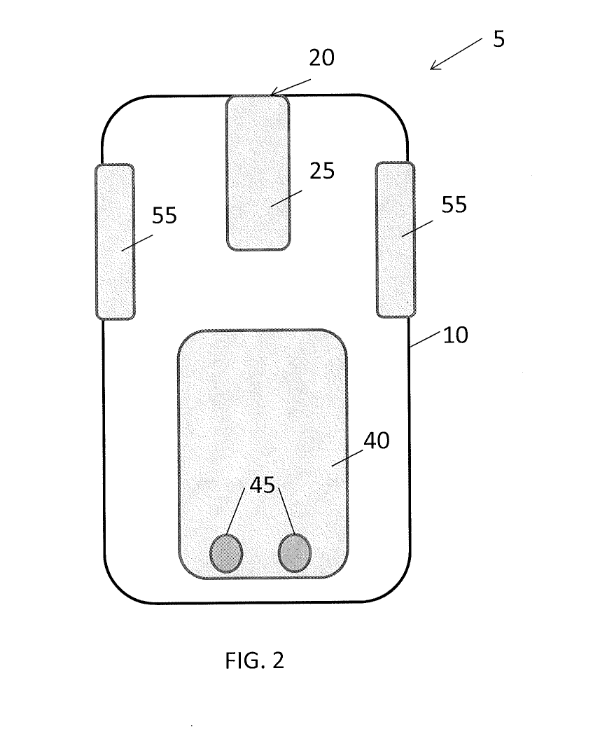

As shown in FIG. 1 the gas meter system 5 can include a housing 10 that can enclose a measuring cell 15 having a plurality of gas sensors 17 configured to detect a concentration of at least two different gases simultaneously. The measuring cell 15 can be operatively coupled to an evaluating circuit 32 on a printed circuit board having a microprocessor 30 and a memory 35. The measuring cell 15 can be in fluid communication with the atmosphere outside the housing 10. The measuring cell 15 can be configured such that the one or more gas sensors 17 can be exposed to ambient air such as by ordinary gas diffusion through one or more openings in the housing 10. Gas can diffuse to the measuring cell 15 from openings on one or more sides of the housing 10. Gas access from more than a single side can prevent obstruction of measuring cell 15. In some implementations, the measuring cell 15 can be positioned near a gas inflow port 20 formed in the housing 10. The inflow port 20 can be covered by a gas permeable membrane 25 to protect the measuring cell 15 from particulate matter in the environment (see FIG. 2). In some implementations, the meter system 5 can be combined with a pump or other negative pressure source to draw gas towards the sensors 17 for remote measurements. It should be appreciated that the configuration of the meter system 5 can vary and is not limited to a particular toxic gas monitoring device. In one implementation, the configuration of the gas meter system 5 can include the portable gas meter described in U.S. Pat. No. 7,395,692 and U.S. Pat. No. 7,336,191, which are incorporated by reference in their entirety herein.

The one or more gas sensors 17 of the measuring cell 15 can include electrochemical substrates, infrared detectors, semiconductor sensors, catalytic sensors or photoionization detectors (PID). Colorimetric gas detection and indication can also be used. Electrochemical gas sensors can detect toxic gases, oxygen deficiency or enrichment or asphyxiate gases. Catalytic bead (pellistor) gas sensors and infrared-optical sensors can detect combustible gases and explosive mixtures. Infrared technology can also be used to measure CO₂. Galvanic gas sensors can detect oxygen. The meter system 5 can include more than a single type of sensor 17. For example, in some implementations the meter system 5 can include a PID sensor and a catalytic or infrared sensor. In some implementations, the sensors can be interchangeably inserted into the housing 10 depending on the type of gas and vapor to be detected by the meter system 5. The sensors can be incorporated into a cartridge type device or removable chip for easy replacement and substitutions. In one implementation, the gas sensors can include those described in U.S. Pat. No. 7,426,849 and U.S. Pat. No. 5,744,697, which are incorporated by reference in their entirety herein.

The measuring cell 15 can detect a variety of gases including, but not limited to, combustible gases and volatile organic compounds in the range of lower explosive limit and methane in the full range, as well as any of the substances listed in Table 1 in the ppm and ppb range. Combustible sensors can measure the flammable substances including and not limited to Ammonia, Ethane, Methane, Pentane, and Propane in the measurement range of 0 to 100% Lower Explosive Limit (LEL).

TABLE 1

| | | Potential |
|---|---|---|
| Gas | Formula | Hazard |
| Ammonia | NH₃ | Flammable |
| Arsine | AsH₃ | Toxic, flammable |
| Carbon dioxide | CO₂ | Asphyxiant |
| Carbon monoxide | CO | Toxic |
| Chlorine | Cl₂ | Toxic, corrosive |
| Ethane | C₂H₆ | Flammable |
| Hydrogen chloride | HCl | Corrosive |
| Hydrogen cyanide | HCN | Toxic |
| Hydrogen fluoride | HF | Toxic, corrosive |
| Hydrogen phosphide | PH₃ | Toxic, flammable |
| Hydrogen sulfide | H₂S | Toxic, flammable |
| Methane | CH₄ | Flammable |
| Nitric oxide | NO | Toxic, oxidizer |
| Nitrogen dioxide | NO₂ | Oxidizer, smog source |
| Ozone | O₃ | Oxidizer |
| Oxygen | O₂ | Oxidizer, flammable |
| Pentane | C₇H₁₆ | Flammable |
| Phosgene | COCl₂ | Toxic |
| Propane | C₃H₈ | Flammable |
| Sulfur dioxide | SO₂ | Toxic |

The microprocessor 30 is in operable communication with the measuring cell 15 and can run a program that carries out the measurement, evaluation and analysis functions, as will be described in more detail below. Limits for combined and individual concentrations of gas species can be programmed to provide a warning to a user according to the Hazard Quotient equation described above. The meter system 5 can be programmed for specific “time-weighted average” (TWA) and “short-term exposure limit” (STEL) categories of threshold limit values, and provide an alarm when either has been exceeded. The sensors can react electrochemically upon exposure to their respective target gas and generate a signal as a result of the reaction. A signal can be sent through the electronics and evaluated by the microprocessor 30. If the signal is higher than a preset threshold value, an alarm or set of alarms can be triggered and implemented. The subsequent actions and reactions can be determined by the specific users of the device.

In an implementation, the apparatus is equipped with two adjustable alarm set points A1 and A2 for a selected measuring range, such as a measuring range of 0-5% CH₄. When the measuring value exceeds an alarm set point an optical and acoustic alarm as well as a vibration alarm are activated. The measuring value can be shown on the display alternating with “A1” at pre-alarm or alternating with “A2” at main alarm, respectively. The main alarm (threshold A2) can be latching, meaning human intervention is needed for the alarm indication to be reset. The optical alarm and the alarm indication on the display cannot be reset for both alarms while the alarm condition is present. The pre-alarm (set point A1) need not be latching. At pre-alarm the acoustic signal as well as the vibration alarm can be switched off while the alarm condition is present.

Various aspects of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, the memory, at least one input device, and at least one output device such as a display.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

As mentioned above, the microprocessor 30 can be operatively connected to a memory 35. The memory 35 can store sensor-specific data such as substance(s) measured, concentration, date, time, temperature compensation, site of measurement, number, calibration values and measure range. The stored data can be retrieved again at any time. The data capacity of the memory 35 can vary. The data capacity can hold the results of a variety of measurements, including 10, 20, 30, 50, 60, or more measurements, together with relevant data. The memory 35 can be volatile and non-volatile, and removable and non-removable. The memory 35 can include computer storage media, including by not limited to RAM, ROM, EEPROM, flash memory or other memory technology, CD-ROM, DVD, or other optical disk storage, or any other medium which can be used to store computer-readable instructions, software, data structures, program modules, and other data which can be accessed by the meter system 5. Data can be accessed directly or through a network such as the internet, WAN or a LAN.

The meter system 5 can include a user interface 40. The user interface 40 can include a visual information display such as an LCD (liquid crystal display), LED, plasma screen, or a CRT (cathode ray tube) for displaying information to the user such as a reading taken by one or more of the sensors 17 or other information. The meter system 5 can also include one or more inputs 45 by which the user may provide input to the circuitry. The input 45 can be received in any form, including acoustic, speech, or tactile input. The input 45 can include user-friendly, mechanical control devices (such as switches, keys, buttons), electrical arrangements (e.g. slider, touch screen), wireless interfaces for communication with a remote controller (e.g. RF, infrared), acoustic interfaces (e.g., with speech recognition), computer network interfaces (e.g., USB port), and other types of interfaces.

The inputs 45 can be used to selectively activate a power supply 50 during a period of interest. The power supply 50 can include a variety of types such as one or more batteries, including disposable or rechargeable batteries. The user interface 40 can indicate the charge of the device if powered by a battery. The meter system 5 can connect to a power charging device.

The meter system 5 can also include an alarm system 55 operatively coupled with the microprocessor 30. The microprocessor 30 can activate the alarm system 55 to notify a user of a measurement or analysis performed on one or more sensed gases. The alarm system 55 can include any form of sensory feedback or alarm (e.g., audible, tactile and/or visual feedback). The alarm system 55 can include one or more illuminated LEDs that indicate a particular status of the meter system 5 and/or the ambient air condition. In some implementations, the LED can illuminate a green color indicating a clean condition of the ambient air. Upon detection of a gas hazard, the LED color can change from green to red. It should be appreciated that other visual warnings can be incorporated. Similarly, a variety of audible warnings or alarms can be incorporated in the meter system 5 such as through a speaker. A triple alarm can also be used in which an audible, visual and tactile alarm can be emitted when the threshold is exceeded or a value falls below a configured concentration. The alarm system 55 can also include a wireless signal (e.g. a wireless transmission to a remote controller or monitor). The meter system 5 can also connect to and operate external alarm equipment such as alarm horns, lamps, traffic lights, etc. remote from the meter system 5. The alarm system 55 can generate one or more alarms using multiple mechanisms simultaneously, concurrently or in a sequence, including redundant mechanisms or complementary mechanisms. Furthermore, hazardous event notifications can be transmitted via various communications protocols including SMS/MMS to individuals within the monitored area as well as to supervisors/control centers overseeing the activities of such individuals. Other notifications can be delivered by other means including voice telephone calls, e-mails, and the like.

The meter system 5 can include a communication system 60 that can send data from the meter system 5 to an external destination or device and vice versa. The communication system 60 can be used to transmit data from the memory 35 to a remote location and/or receive data from remote location device. The meter system 5 in turn can provide real-time warnings of substances detected in an area. The data can be downloaded through the communication system 60 to a remote or local PC, laptop, communication station, another detector system, or other remote device, over a variety of communication lines such as by wired or wireless connection, RF, IR, optical, and others.

The meter system 5 may be periodically calibrated to ensure the readings generated are accurate. The meter system 5 can be provided with a known concentration of a known substance and the meter system 5 reading adjusted to reflect the known concentration of the known substance or known concentrations of a combination of known substances. The sensors of the meter system 5 can be calibrated on a regular basis specific to each sensor type, varying generally from 1 to 12 months. The sensors can be challenged, such as by a function test or a “bump” test, with a known concentration of gas that exceeds alarm set thresholds for each sensor before any use.

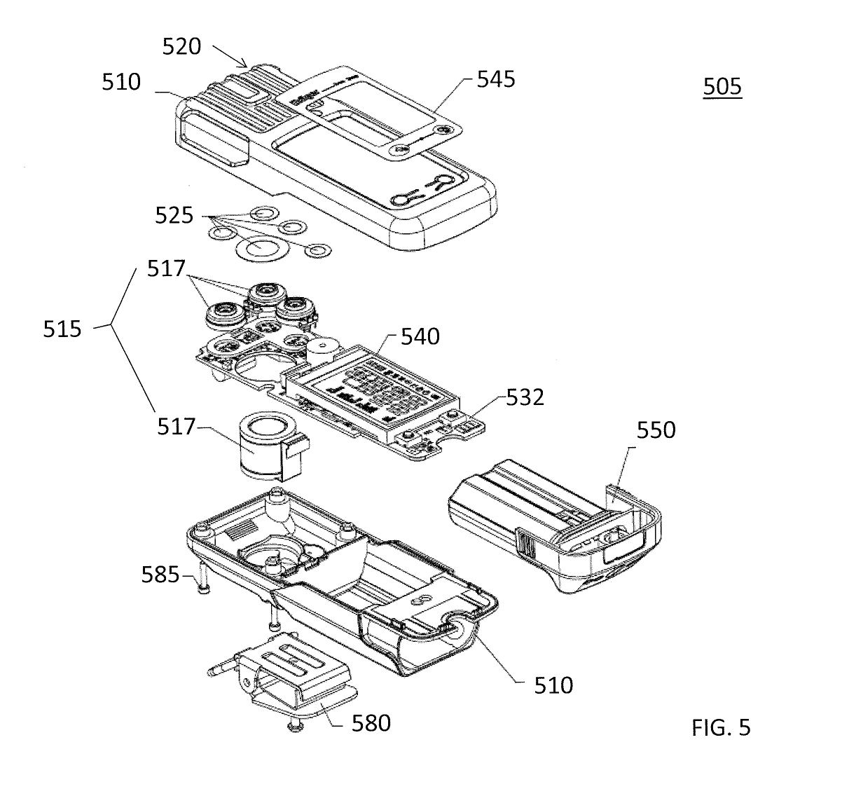

The meter system 5 can be suitable for both mobile and stationary use. In some implementations, the meter system 5 can be a portable or mobile system, such as a hand-held system or a system capable of being carried by a person of ordinary strength. The meter system 5 can be small enough to be clipped onto a person, such as on a belt or piece of clothing using a clip accessory 580 coupled to a portion of the housing 10 (see FIG. 5). Alternatively, the meter system 5 can be held and transported using a handle coupled to a portion of the housing 10.

In some implementations, one or more mobile gas meter systems 5 can be used in combination with a base station or an area monitoring device 1 (see FIG. 3). The area monitoring device 1 can be stationary or mobile. The area monitoring device 1 and gas meter systems 5 can each perform two-way communication 7 with one another. In some implementations, the multiple gas meter systems 5 can detect a combination of gases and communicate the raw data of the readings to the area monitoring device 1, which in turn can perform the calculations and evaluations of the readings to determine whether a hazard exists in one or more of the areas. In other implementations, the multiple gas meter systems 5 can detect a combination of gases, perform the calculation and evaluation of the raw data of the readings, and communicate the evaluation to the area monitoring device 1. In further implementations, one of the gas meter systems 5 can sense one type of gas and a second gas meter system 5 can sense a second different type of gas. Each meter system 5 can communicate their respective raw data to the area monitoring device 1, which can evaluate the data and perform necessary calculations to determine whether a hazard condition exists. Examples of area monitoring systems are described in U.S. Pat. No. 7,336,191, which is incorporated by reference in its entirety.

The meter system 5 can be programmed by a user. The desired Threshold Limit Values (TLV) for individual gases can be defined prior to use with the one or more inputs 45 based upon the likelihood of those gases to be detected in a particular environment. Alternatively, the Threshold Limit Values (TLV) can be pre-programmed. All settings can be pre-programmed to factory default settings for typical use and generally accepted parameters. The user can adjust these settings for specific applications, special conditions and/or local requirements. The user can perform the adjustments by accessing menus in the device or communicating to the device via an interface such as a PC or other device. Parameters such as alarm set points, test gas settings, calibration gas concentrations, logging frequency, types of gases monitored and other parameters can be set to user preferences from the factory defaults.

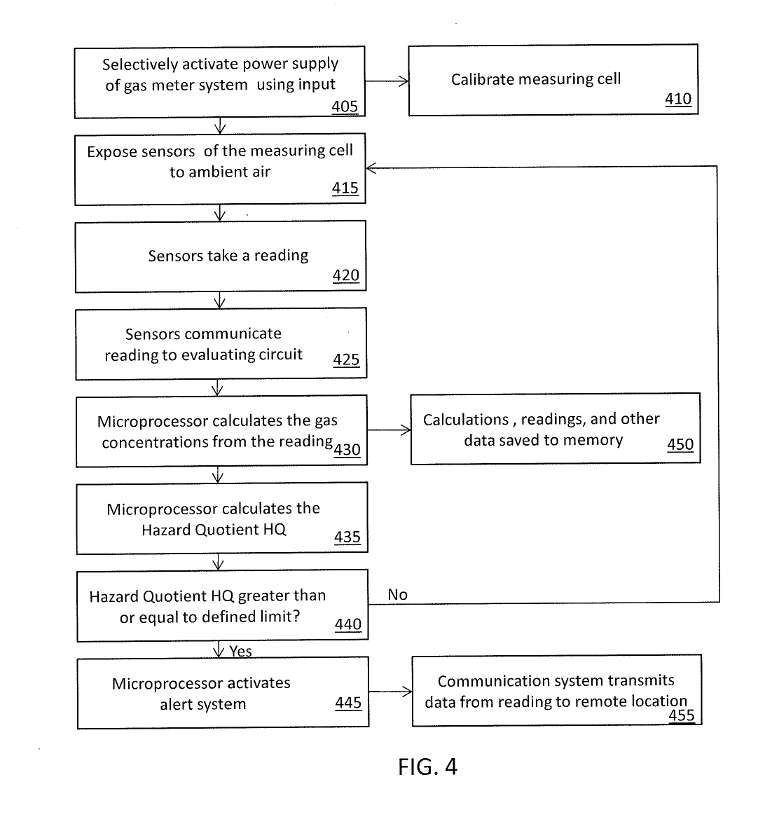

FIG. 4 illustrates an implementation of a method of use of a gas meter system 5. A user can selectively activate the power supply 50 of the gas meter system using an input 45 (405). The user can select a calibration of the measuring cell 15 (410). The one or more sensors 17 of the measuring cell 15 can be exposed to the ambient air (415). A reading can be taken (420) and the raw data communicated to the evaluating circuit 32 (425). In some implementations, the readings can be taken continuously. In some implementations, the readings can be “spot” readings and taken according to a scheduled interval. In some implementations, the device incorporates a pump or other device that can move air towards the device to allow different areas of an environment to be read. The microprocessor 30 runs the software program to calculate the gas concentrations (430) and the Hazard Quotient (HQ) for the combination of substances detected (435). The software program can evaluate whether a potential hazard exists based on whether the Hazard Quotient (HQ) is greater than the pre-set defined value (440). If the HQ is greater than or equal to the defined limit, the microprocessor 30 activates the alarm system 55 (445). If the HQ is not greater than or equal to the defined limit, no alarm system 55 is activated. The data associated with the readings can be saved to memory 35 (450). The microprocessor 30 can also activate the user interface 40 to provide a user with information regarding the analysis performed by the microprocessor 30 including for example, the substance(s) detected, substance(s) concentration, Hazard Quotient (HQ), status of environment, etc. The microprocessor 30 can also activate the communication system 60 to transmit the data from the meter system 5 to a remote location such as a monitoring station (455).

FIG. 5 illustrates an exploded view of an implementation of a gas meter system 505 having a removable and replaceable gas sensor 517. The gas meter system 505 can include a two-part housing 410 that can enclose a measuring cell 515 having a plurality of gas sensors 517. The measuring cell 515 can be operatively coupled to an evaluating circuit 532 positioned on a printed circuit board having a microprocessor and a memory that can interface with a display 540 and programmed using an input 545. The measuring cell 515 can be in fluid communication with the atmosphere outside the housing 510 and configured such that the sensors 517 can be exposed to ambient air through one or more openings 520 in the housing 510. The openings 520 can be aligned with the sensors 517 and have a gas permeable membrane 525 positioned in between to protect the measuring cell 515 and sensors 517 from particulate matter in the environment. The housing 510 can be reversibly fastened such as by one or more fasteners 585 such as Allen screws or other fasteners. This allows for one or more of the enclosed sensors 517 to be accessed by a user, removed from the housing 510 and replaced by another appropriate sensor 517.

The implementations set forth in the foregoing description do not represent all implementations consistent with the subject matter described herein. Instead, they are merely some examples consistent with aspects related to the described subject matter. Wherever possible, the same reference numbers will be used throughout the drawings to refer to the same or like parts.

Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows and steps for use described herein do not require the particular order shown, or sequential order, to achieve desirable results. Other embodiments can be within the scope of the claims.

1. A gas meter system for detecting toxic gas combinations, the system comprising:
a housing having an opening;
a measuring cell enclosed by the housing and comprising a plurality of gas sensors in fluid communication with the environment via the opening in the housing, the gas sensors detecting a concentration of a first gas and a concentration of a second gas; and
an evaluating circuit to identify a hazard due to an additive toxic effect upon combined exposure to the first gas and the second gas.

2. The system of claim 1, wherein the additive toxic effect is identified by a Hazard Quotient (HQ) equation:
HQ=C_{G1}/TLV_{G1}+C_{G2}/TLV_{G2}
wherein C_{G1} is the first gas concentration; TLV_{G1} is a desired threshold limit value of the first gas; C_{G2} is the second gas concentration; and TLV_{G2} is a desired threshold limit value of the second gas.

3. The system of claim 2, wherein a hazard is identified when the HQ is greater than or equal to a defined hazard limit.

4. The system of claim 3, wherein the hazard is identified when at least one of the first gas concentration and the second gas concentration is below its respective defined threshold limit value.

5. The system of claim 4, wherein the hazard is identified when both the first gas concentration and the second gas concentration are below their respective defined threshold limit values.

6. The system of claim 3, further comprising an alarm system to be activated when the hazard is identified.

7. The system of claim 6, wherein the alarm system comprises at least one of an audible, visual, or tactile alarm.

8. The system of claim 1, wherein the first gas and the second gas are different gases.

9. The system of claim 1, wherein the first gas is carbon monoxide and the second gas is hydrogen cyanide.

10. The system of claim 1, wherein one or more of the gas sensors are selected from the group consisting of an electrochemical, infrared, semiconductor, catalytic, photoionization, and galvanic sensor.

11. (canceled)

12. A method of detecting an additive effect of toxic gases, comprising:
sensing a concentration of a first gas in an environment using a gas meter system, wherein the gas meter system includes a microprocessor having a programmed first desired threshold limit value of the first gas;
sensing a concentration of a second gas in the environment using the gas meter system, wherein the microprocessor of the gas meter system has a programmed second desired threshold limit value of the second gas;
providing the sensed concentrations of the first and second gases to the microprocessor;
using the microprocessor to calculate a Hazard Quotient (HQ) using equation:
HQ=C_{G1}/TLV_{G1}+C_{G2}/TLV_{G2}
wherein C_{G1} is the concentration of the first gas; TLV_{G1} is the first desired threshold limit value of the first gas; C_{G2} is the concentration of the second gas; and TLV_{G2} is the second desired threshold limit value of the second gas; and
evaluating whether the HQ is greater than or equal to a defined hazard limit programmed into the microprocessor.

13. The method of claim 12, further comprising identifying a hazard when the HQ is greater than or equal to the defined hazard limit.

14. The method of claim 13, wherein the hazard is identified when the concentration of at least one of the first gas and the second gas is below its respective defined threshold limit value.

15. The method of claim 14, wherein the hazard is identified when both the first gas concentration and the second gas concentration are below their respective defined threshold limit values.

16. The method of claim 13, further comprising activating an alarm system when the hazard is identified.

17. The method of claim 16, wherein activating the alarm system comprises at least one of an audible, visual or tactile alarm.

18. The method of claim 12, wherein the first gas and the second gas are different gases.

19. The method of claim 12, wherein the first gas is carbon monoxide and the second gas is hydrogen cyanide.

20. The method of claim 12, wherein sensing a concentration of the first gas comprises using one or more gas sensors selected from the group consisting of an electrochemical, infrared, semiconductor, catalytic, photoionization, and galvanic sensor.

21. The method of claim 12, wherein sensing a concentration of the second gas comprises using one or more gas sensors selected from the group consisting of an electrochemical, infrared, semiconductor, catalytic, photoionization, and galvanic sensor.

22. A gas measuring system comprising:
a first gas meter device configured to detect a concentration of a first gas within a monitored zone;
a second gas meter device separate and apart from the first meter device configured to detect a concentration of a second gas the monitored zone;
a base station in communication with the first gas meter device and the second gas meter device, the base station calculating an additive toxic effect by a Hazard Quotient (HQ) equation:
HQ=C_{G1}/TLV_{G1}+C_{G2}/TLV_{G2}
wherein C_{G1} is the first gas concentration; TLV_{G1} is a desired threshold limit value of the first gas; C_{G2} is the second gas concentration; and TLV_{G2} is a desired threshold limit value of the second gas, the base station initiating an alarm when the additive toxic effect is above a pre-defined hazard limit.

23-26. (canceled)
